(19) United States
(12) Patent Application Publication
(10) Pub. No.: US 2020/0368524 A1
(43) Pub. Date: Nov. 26, 2020

(54) Electrode Placement System for Penetrating Neural Implants

(71) Applicant: Actuated Medical, Inc., Bellefonte, PA (US)

(72) Inventors: Roger B. Bagwell, Bellefonte, PA (US); Ryan S. Clement, State College, PA (US); Natasha N. Tirko, State College, PA (US); Jenna K. Greaser, State College, PA (US)

(73) Assignee: Actuated Medical, Inc., Bellefonte, PA (US)

(21) Appl. No.: 16/847,294

(22) Filed: Apr. 13, 2020

Related U.S. Application Data
(60) Provisional application No. 62/851,235, filed on May 22, 2019.

(51) Int. Cl. A61N 1/05; A61B 17/32

(52) U.S. Cl. CPC A61N 1/0551; A61B 2017/00398; A61B 17/320068

(57) ABSTRACT

An insertion assembly for inserting a neural implant into target neural tissue. The insertion assembly includes a horn connecting to a vibrational actuator. A horn tip is configured to support an implant for insertion, such as the base of an implant having electrode(s). An implant stabilizer is displaceably affixed to the horn at a connection point for secure mounting while permitting flexing of the implant stabilizer. The implant stabilizer includes a first end positionable proximate to the horn tip and contacting the implant, and an opposite second end that may be selectively moved by force to adjust the position of the first end relative to the horn tip. A biasing member contacts the horn tip and first end of the implant stabilizer and urges the first end toward the horn tip to releaseably secure the implant to the horn tip. The biasing member may be selectively removed from the assembly.

### CROSS-REFERENCE TO RELATED APPLICATIONS

The present application claims priority to U.S. Provisional Application Ser. No. 62/851,235 filed on May 22, 2019, the contents of which are incorporated herein by reference in its entirety.

### STATEMENT REGARDING FEDERALLY SPONSORED RESEARCH OR DEVELOPMENT

This invention was made with government support under HR0011-16-C-0094 awarded by the Defense Advanced Research Projects Agency; under NS105500 awarded by the National Institutes of Health/National Institute of Neurological Disorders and Stroke; and under DK120349 awarded by the National Institutes of Health/National Institute of Diabetes and Digestive and Kidney Diseases. The government has certain rights in the invention.

### FIELD OF THE INVENTION

The present invention relates to the insertion of neural implants which contain one or more electrodes arranged on one or more penetrating members, such as shanks, that are inserted into neural tissues for the purpose of establishing a neural interface through which to directly record and/or to stimulate neural activity. More specifically, the invention relates to a system to aid the insertion of neural implants through application of high frequency micro-vibration to the penetrating member(s) of the probe or electrode array to reduce the forces required to penetrate the tissue. The system also includes target stabilization mechanisms utilized during insertion for precise implant placement.

### BACKGROUND

Neural implants, such as chronically implanted microelectrode arrays designed to interface with neural tissue hold great potential for revolutionizing treatment of a range of medical conditions. Applications of neural implants include neural-based control of prosthetic limbs by amputees, brain-machine interfacing for paraplegics, selective ablation and/or inactivation of problematic neural pathways, or control or enhancement of organ function. Programs like SPARC, the BRAIN Initiative, and BrainGate are bringing new neuroprosthetic devices to patients, and researchers predict that neural implants will be more widely implemented in humans in the next 10 years. Non-penetrating neural implant electrode arrays such as EEG electrodes and nerve cuffs have seen increased clinical application recent years, but such systems have limited spatial resolution, making them less ideal for future applications requiring more precise stimulation or recording. Penetrating neural electrode arrays offer significantly improved temporal and spatial resolution but suffer from multiple complications which restrict their clinical use. A major complication is the limited ability to precisely position the electrode array's penetrating members, or shanks, in the desired location, which is exacerbated by tissue compression, particularly when the electrode array consists of multiple closely spaced penetrating members, as in the “bed of nails” designs of the Utah (Blackrock) or microwire electrode arrays. The mechanical stress of implantation may also lead to penetrating member damage or bending and deflection that further exacerbates the tendency to miss the desired target or fail to penetrate the tissue altogether.

Additionally, the trauma of implantation, including the dimpling of local tissue and nerves, may decrease recording yield and can cause and/or accelerate glial scarring which isolates the implant from the target tissue. Chronically placed neural penetrating members cause a reactive tissue response involving astrocytes and microglia that result in the formation of a cellular sheath or scar around the penetrating member. The response is highly complex with multiple chemical signaling pathways, cell types, and damage involved, but overall involves an initial acute phase of glial scarring in response to the initial injury followed by chronic inflammation. Previous studies comparing fast and slow insertion speeds have found that both electrophysiological and histological outcomes are more favorable with faster insertion so the faster insertion is often the approach typically used. Studies exploring the role of the electrode array density are largely lacking in the literature. However, given the fact that gliosis can often extend 500-600 μm beyond the implant-tissue interface, it is likely that there is compounding interaction among penetrating members that have overlapping regions of influence. In addition, more densely packed penetrating members will likely increase the implantation trauma and dimpling.

As a subset of neural implants—penetrating intracortical microelectrode arrays—are composed of multiple penetrating members with typical cross-sectional diameters in the range of 25-100 μm and are typically implanted 0.25-2 mm into brain tissue, but sometimes as deep as several centimeters when targeting deep brain structures in some animal species. The recording sites are relatively small with high impedance (>100 kΩ), a requirement for recording unit activity from individual neurons. Variations in penetrating electrode technologies include insulated metallic microwires, micromachined high density 3-D electrode arrays such as Utah electrode array that are similar in geometry to microwire electrode arrays, and planar thin-film microelectrode arrays like Michigan probes, also known as NeuroNexus, composed of silicon or polymer substrates with multiple electrode sites along the penetrating members. In addition to material and method of fabrication considerations, penetrating electrode designs may differ in: (a) geometry, including but not limited to tip shape, size and spacing of the penetrating members: (b) attachment state relative to the neural target, such as fixed or floating; and (c) insertion strategy, including by hand, manually with a micromanipulator, pneumatic impact, or mechanized insertion at fast or slow speeds. As the density of penetrating members of the electrode array increases, it is more likely to dimple or compress the neural tissue during implantation. One strategy employed for implantation of Utah electrode arrays in brain and nerve tissue is to use a pneumatic, single-shot, high speed impact inserter to essentially hammer the implant into neural tissue at high velocity in order to reduce dimpling. Since the inserter only makes momentary contact with the electrode array, this single shot approach does not allow for fine adjustment or correction if the initial placement is not ideal, or when fine anatomic details vary across subjects. Successful insertion is still often heavily reliant on surgical skill and technique.

Most types of neural microelectrode arrays, including microwires, 3-D silicon, and 2-D planar silicon devices, have published examples demonstrating the ability to record neural activity upwards of a year or more in many different subjects. However, the consistency in performance of penetrating neural microelectrode arrays is highly variable. For instance, a group at University of Michigan now has a team of individuals experienced in implanting their microelectrode arrays in subjects, and approximately 67% of the time the implants record unit activity for 3-6 months or more. However, the remaining 33% of the electrode arrays often fail at around 6 weeks, suggesting that if the microelectrode arrays can make it beyond this critical window, they could record neural activity indefinitely. According to an informal survey by Schwartz, any given recording electrode site on a penetrating member may only have a 40-60% chance of recording chronic neural activity and essentially all conductive penetrating members do eventually fail.

Therefore, a way to insert penetrating electrodes into neural tissue in a manner that preserves the integrity of the electrodes and minimizes damage and trauma to the surrounding neural tissue is still needed, for increased accuracy of placement and long-term use of the resulting embedded electrodes.

Peripheral neural targets like the dorsal root ganglia, nerves, spine, and even muscle tissue in the arms and legs present and even greater challenge for penetrating neural implant placement than that encountered for relatively soft tissue in the cortex of the brain. Compared to the brain, penetrating microelectrode array technology has been largely under-utilized in both basic and applied peripheral nervous system research. There are numerous reasons for this including greater difficulty in accessing and stabilizing the neural targets during surgery and challenges associated with either dissecting, or getting the shanks to penetrate, the neural membranes (epineurium or dura). There is increasing interest however in achieving direct interfacing with neural tissues outside the brain and with an approach that reduces tissue damage and improves implant location accuracy, allowing placement for instance in or near specific fascicles or neural circuits. As an example, the urinary system is a target for placement of penetrating neural implants where a great clinical need exists. Neurogenic bladder dysfunction, or the interruption in neural communication between the control circuit and bladder muscles, occurs in a staggering 70-84% of spinal cord injury patients. After spinal cord injury, lower urinary tract dysfunction generally presents as a reflexive bladder and sphincter paralysis. This is of particular concern in the military veteran population, where an estimated 32,000 spinal cord injury veterans suffer from micturition disorders. Inadequate post-injury management of lower urinary tract dysfunction can lead to complications from infection to total renal failure, which was previously the leading cause of death after spinal cord injury.

The ability to store and eliminate urine is regulated by a dynamic neural circuit integrating information from brain, spinal cord and peripheral autonomic ganglia. The lower urinary tract coordinates activity between smooth and striated muscles in the bladder and urethral outlet, to both store urine and void it. Numerous interventions attempt to treat neurogenic bladder dysfunction: timed voiding, manual expression, medications, catheterization (both intermittent and indwelling), and surgical procedures. The current clinical standard of care for neurogenic bladder dysfunction patients remains catheterization, however all forms of catheterization are associated with risk of infection, which causes these patients an average of 16 office and 0.5 emergency room visits per year and possible hospitalization. Alternative treatments to restore function of the neurogenic bladder system have been developed with varying degrees of success. in addition to catheterization, mechanical solutions such as artificial urethral sphincters, stents, and pumps have been tested, however all have similar risks of infection and limited functional lifespans. Pharmacology treatments like anticholinergic medications can also be used to relax the hyper-reflexive bladder but have systemic side effects like dry mouth and blurred vision.

Electrical control of the bladder through neuroprosthetics would avoid the inconvenience, recurring cost, and associated infection risk of catheterization, as well as the systemic problems from pharmacological drug herapies. The effectiveness of electrical stimulation devices is limited by a number of factors including the stimulation target, the type of stimulating electrode, surgical access to nerves, and device longevity. However, the effectiveness of existing electrical stimulation devices is restricted by limited surgical access to the nerves, difficulty in electrode placement, and poor stimulation specificity. Initial attempts at a nerve-based bladder control device relied on non-penetrating electrode technologies such as nerve cuffs that excite a large portion of the pudendal nerve, but this approach requires spatially segregated stimulation to avoid simultaneous activation of antagonistic muscle groups of the bladder. Penetrating multichannel electrodes allow more spatially specific activation of nerve fibers that could significantly improve outcomes. However, implantation of penetrating electrodes into nerves remains a great challenge. Piercing the epineurium requires the electrode to withstand forces which may buckle or break the electrode. In addition, nerves typically compress (dimple), stretch, and/or roll, which prohibits effective electrode insertion, increases risk of trauma, bleeding and inflammation to the nerve tissue, and may accentuate the chronic foreign body response (FBR) leading to cell death, peripheral nerve scaring, and device failure. For clinically viable chronic penetrating nerve interfaces, the insertion forces must be substantially reduced and the nerve must be better stabilized during electrode insertion process in a way that is both minimally invasive and temporary.

Multiple stimulation targets have been examined as potential sites for restoration of urinary function, with varying success. These include electrical stimulation of the bladder, transcutaneous electrical stimulation of various nerves, stimulation of sacral roots and nerves, stimulation of the spinal cord, and stimulation of peripheral nerves. The latter two approaches require highly invasive surgical procedures and have failure rates as high as 40%. For instance, stimulation of the pudendal nerve with a surface linear electrode, laparoscopically placed in soft tissue adjacent to the nerve has been performed. This procedure successfully controlled micturition in patients with overactive bladder but the low-resolution stimulation of the entire pudendal nerve bundle was insufficient for treatment of other types of bladder dysfunction. The pudendal nerve is a particularly good target for human bladder control, having consistent fascicular anatomy between individuals. An alternative extraneural electrode device, called BION, has shown some success, though technical failures and migration of the electrodes once embedded prohibited reliability. To improve stimulation specificity and more stable interface for longevity of the electrical stimulation effectiveness, an intraneural penetrating electrode solution is necessary. A promising approach with potential for future clinical use would involve peripheral nerve stimulation, but only with improved surgical approach and utilizing penetrating electrode arrays.

Intraneural, or penetrating microelectrodes are placed directly in the peripheral nerve and solve several problems presented by cuff and other extraneurally placed electrodes for interfacing with peripheral neural targets. Penetrating microelectrodes can be longitudinally implanted or transversely implanted, offering different surgical implantation strategies. Penetrating microelectrodes grant specificity of stimulation, as individual electrode surfaces and combinatorial activation of electrode pairs can target independent fascicles. Peripheral nerves also have considerable freedom of movement as compared to brain or spinal cord, so penetrating electrodes can also be more resistant to migration, as they are embedded in the nerve and more likely to move with the nerve as it may move in the body when surrounding tissues are stretched.

Without a means of stabilization during the insertion of penetrating neural implants, the nerves can stretch and roll which may prevent complete penetration or result in inaccurate placement of the implant. Furthermore, it is important for the orientation and trajectory of the penetrating members of the neural implant to remain parallel to the insertion axis and for the axis to remain constant relative to the neural target to prevent inaccuracy as well as to minimize the extent of damage to neural tissue adjacent to insertion tract. For peripheral neural targets, which are not as confined relative to rigid bone as the brain is, the insertion of penetrating neural implants is not straightforward and not readily available in the marketplace.

Another critical aspect for insertion of penetrating neural implants, whether for the central or peripheral nervous system, is the ability to securely grasp a wide range of implant shapes and styles. The implants, which are very small, often sub-millimeter dimensions on each side, need be firmly grasped so they do not move out of alignment with the insertion axis as they experience forces during penetration process. At the same time, the devices are often very fragile and extremely expensive to manufacture, so the means of grasping them needs to be gentle and not apply excessive mechanical forces that may crush, bend, or otherwise break the implant. Furthermore, the implant also needs to be easily released when it is positioned in the desired location in the tissue, without having to twist, turn, or apply excessive torque to the implant that may apply forces to the surrounding tissue or inadvertently move the implant out of the desired position.

A major contribution to the failure of electrodes over time is believed to be the stiffness mismatch between the tissue target and the neural implant, which can induce a damaging tissue response exacerbated by relative motion between the neural implant and surrounding neural tissue. Thinner, more flexible implants are therefore desirable but will be much more difficult to insert through tough peripheral neural targets. The challenge of inserting any penetrating microelectrodes, particularly flexible ones, is overcoming the penetration force of the tough epineurium layer around peripheral nerves, or the meningeal membranes of brain and spinal cord.

There is still much room for improvement in the field of electrode use for neurological stimulation, particularly in the areas of penetrating electrodes, floating arrays that are not anchored to bone, and in-dwelling or embedded implants that remain resident in the tissue for extended periods of time.

### SUMMARY

An electrode placement system is disclosed for the accurate and precise insertion and placement of penetrating electrodes into neural tissue, such as any tissues of the nervous system, including but not limited to the brain(including the cortical brain as well as deeper brain structures), spinal cord, dorsal root ganglion, peripheral nerves and peripheral nerve bundles. The system provides enhanced placement accuracy and functionality of penetrating nerve electrodes by stabilizing the neural target while vibrating the penetrating electrode during insertion, reducing insertion force and increasing insertion success while reducing strain and trauma to the neural tissue. As a result, this system improves implant location accuracy, allowing placement in and near specific cortical targets and/or nerve fascicles for highly specific electrode placement, neural stimulation and recording of neural activity.

The system of the present invention can be for the insertion and placement of any type of penetrating electrode, including stimulating and recording electrodes. The penetrating electrodes may be part of an implant or array, which can include a single or multiple penetrating members. The implants may be made of many different types of biocompatible materials, including but not limited to microwire, silicon, carbon fiber, optical fiber, and/or polymers and various composites The system can also be used to facilitate the insertion of thinner and more flexible implants and electrodes than current insertion options allow for. The current invention reduces the insertion force, implant buckling and breaking, and tissue dimpling to allow improved neural interface establishment. Furthermore, reducing the insertion force facilitates the insertion of thinner and more flexible penetrating electrodes because the buckling force requirements are reduced. Longer electrodes and probes can be more successfully inserted to a target location due to reduced buckling and deflection.

The system of the present invention may be hand-held in some embodiments, tabletop mounted such as on a stereotaxic frame in other embodiments and may be deployable through a trocar or laparoscope for clinical use and minimally invasive procedures in some embodiments.

The electrode placement system of the present invention includes a vibrational actuator configured to generate and deliver micro-vibrations to an implant having one or more penetrating members or shanks. The vibrations are provided axially along the insertion axis of the implant to oscillate the implant axially during insertion of the penetrating member(s) into target neural tissue. A translational motor is also included in the system to move the implant linearly along the insertion axis, to advance the penetrating electrode(s) along a desired path at a controlled speed into the target neural tissue and to release and retract the supporting components of the system once the implant is embedded in the target neural tissue. The system also includes a control unit with a processor and vibrational and translational drivers. The vibrational actuator and translational motor are each in electrical communication with the control unit and receive operative instructions from the respective drivers to activate and operate.

The system may also include a target stabilization assembly that is configured to contact and hold the target neural tissue in place during the insertion process, The target stabilization assembly provides mechanical stability of the target neural tissue and establishes a point of reference from which the system inserts the neural implant to the location of the desired neural target. It also limits the movement and stretch of the target neural tissue with minimal dissection The target stabilization assembly includes at least one arm having at least one finger at a terminal end. The arm(s) and finger(s) may be manipulated to engage the target neural tissue, either directly or through other anatomical structures such as the skull, such as an ear canal, and hold it in position for electrode insertion. In at least one embodiment, the arm(s) and finger(s) are selectively movable relative to one another, such as in the longitudinal direction parallel to the insertion axis of the implant, to grip a portion of the target neural tissue therebetween. The target stabilization assembly may also apply slight pressure to the target neural tissue, such as by pulling, pressing or vacuum, to increase the tautness of the surface of the target neural tissue and further limit movement of the target site.

The electrode placement system also includes an insertion assembly configured to selectively retain and release the implant having at least one penetrating member. The insertion assembly includes a horn that is connected to or part of the vibrational actuator and transmits the axially-directed vibrations to the implant and electrode(s) during insertion. The horn tip located opposite from the vibrational actuator is shaped to contact and abut the implant, and may have a recess formed therein in certain embodiments correspondingly shaped to receive a portion of the implant therein for increased stabilization, The insertion assembly also includes an implant stabilizer of stiff yet bendable material that is displaceably mounted to the horn at a connection point. A first end of the implant stabilizer extending from the connection point is positioned proximate to the horn tip and is configured to contact and hold the implant against the horn tip in tight enough contact for effective vibration transfer. A second end of the implant stabilizer extends from the opposite side of the connection point and is selectively movable under the application of force, causing the first end to also move relative to the horn tip. A biasing member contacts the first end of the implant stabilizer and the horn tip and urges the first end of the implant stabilizer d the horn tip to provide force on the implant for retention and vibrational energy coupling to the implant. Application of force to the second end of the implant stabilizer moves the first end in a direction away from the horn tip, limited by the biasing member but sufficient enough to release the grip on the implant for loading or release of the implant. The biasing member may also be selectively removed from the insertion assembly, such as by cutting, when the penetrating electrode(s) of the implant are inserted to the desired target without perturbing the electrode(s) so the insertion assembly may be removed, leaving the implant embedded in place in the target site.

The electrode placement system, together with its particular features and advantages, will become more apparent from the following detailed description and with reference to the appended drawings.

### DESCRIPTION OF THE DRAWINGS

FIG. 1 is a schematic diagram of an overview of the electrode placement system of the present invention.

FIG. 2A is a perspective view of one embodiment of the system utilizing a stereotaxic frame and pins for target stabilization.

FIG. 2B is a detailed view of the insertion site of the target stabilization apparatus of FIG. 2A.

FIG. 3 is a side elevation view of a second embodiment of the electrode placement system that is handheld and utilizes longitudinal gripping arms for target stabilization.

FIG. 4 is a partial cutaway view of the insertion device of FIG. 3 with the gripping arms extended.

FIG. 5A is a detail view of the distal end of the gripping arms prior to target stabilization and electrode insertion.

FIG. 5B is a detail view of the distal end of the device of FIG. 5A with the target nerve stabilized and the electrode inserted.

FIG. 6A is a side elevation view of another embodiment of the insertion device that is handheld and utilizes a single gripping arm for target stabilization.

FIG. 6B is a side elevation view of the insertion device of FIG. 6A showing gripping of neural tissue.

FIG. 7 is a diagram of another embodiment of the insertion device utilizing vacuum for target stabilization.

FIG. 8 is a detail view of the target stabilization apparatus of FIG. 7.

FIG. 9A is a top plan diagram of the target stabilization apparatus of FIG. 7.

FIG. 9B is a top plan diagram of a second embodiment of the target stabilization apparatus utilizing vacuum for stabilization.

FIG. 10 is a schematic diagram of a third embodiment of the target stabilization apparatus utilizing vacuum.

FIG. 11 is a schematic diagram of a fourth embodiment of the target stabilization apparatus utilizing vacuum.

FIG. 12 is a side elevation view of one embodiment of the implant stabilizer of the present invention.

FIG. 13A is a detail perspective view of one embodiment of the first end of the implant stabilizer of FIG. 12 having a solid configuration.

FIG. 13B is a detail perspective view of another embodiment of the first end of the implant stabilizer of FIG. 12 having an aperture to accommodate the cable to pass from the implant to pass therethrough.

FIG. 14A is a diagram of the insertion assembly of FIG. 12, showing the loading of the implant.

FIG. 14B is a diagram of the insertion assembly of FIG. 12, showing securing the implant in the implant stabilizer.

FIG. 14C is a diagram of the insertion assembly of FIG. 12, showing removal of the biasing member.

FIG. 14D is a diagram of the implant stabilization apparatus of FIG. 12, showing removal of the insertion assembly from the embedded implant.

Like reference numerals refer to like parts throughout the several views of the drawings.

### DETAILED DESCRIPTION

As shown in the accompanying drawings, the present invention is directed to an electrode placement system 100 for the precision insertion and embedding of electrodes into neural tissue 5. The system 100 can be used to insert one or more electrode penetrating members 122, either singly or in a combined implant 121, into a desired neural target. As used herein, the term “implant” may refer to a penetrating electrode array, which is inserted and anchored in the neural tissue 5, bringing the electrode sites closer to the underlying neurons while restricting the migration of the implant relative to the neural tissue as might occur with a non-penetrating extra-neural implant. Penetrating electrode arrays therefore allow specificity of stimulation, as individual electrode surfaces can target independent fascicles and/or neural circuits. Activation of opposing functions may be avoided if appropriate populations of nerve fibers within individual fascicles, or specific neurons of a circuit or center, can be selectively targeted. The neural tissue targeted for insertion and electrode implantation may be any neural target, including but not limited to brain tissue (including cortical and/or deep brain structures), the spinal cord, and peripheral nerves. The system 100 utilizes oscillation vibrations in the ultrasonic frequency range to reduce the force required for insertion and implantation of electrode arrays and to reduce the dimpling of the soft tissue that is being penetrated. The objective of the system 100 is to improve insertion success while reducing strain and trauma to recipient tissue.

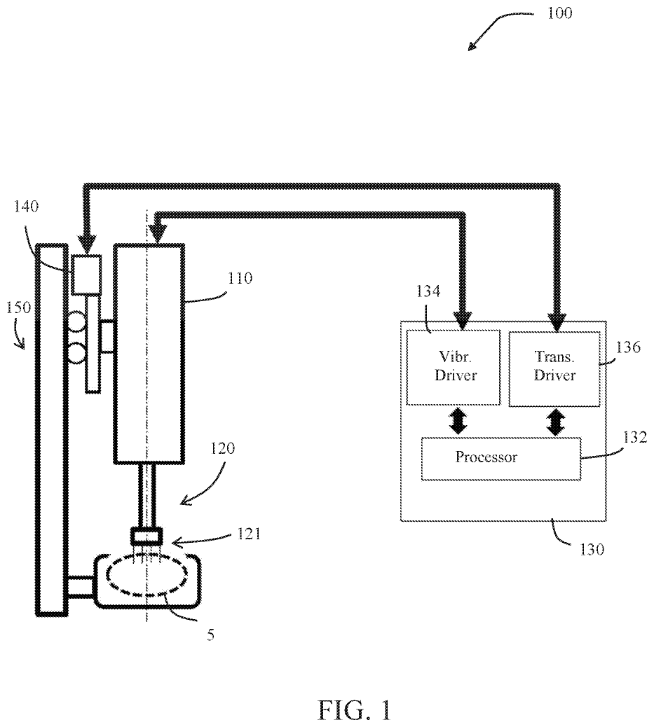

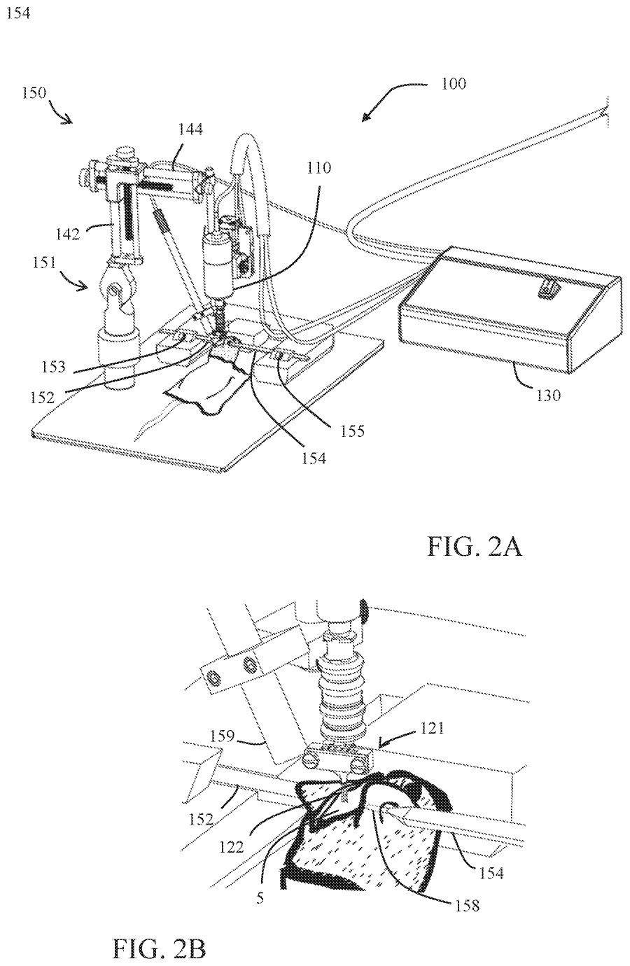

The electrode placement system 100 includes a vibrational actuator 110, an insertion assembly 120, a control unit 130, a translational motor 140 and a target stabilization assembly 150, as depicted in FIG. 1. The system 100 may be a tabletop unit in some embodiments, such as shown in FIG. 2A, or it may be a handheld unit in other embodiments such as shown in FIGS. 3, 4, 6A-6B and 7. The system 100 may be used to insert any neural implant 121, including but not limited to multi-channel, single-shank device like Modular Bionics N-Form probes; arrays 121 having multiple penetrating members 122 including but not limited to NeuroNexus, BlackRock; microwire arrays such as but not limited to those manufactured by Tucker-Davis Technologies or MicroProbes for Life Sciences. The implant 121 may be a fixed array that is affixed to the skull, bone, or other rigid material surrounding the target neural tissue, a tethered array on the end of a flexible cable 125 which is anchored elsewhere, or a completely floating array that is embedded in the target neural tissue but are not affixed to any other material and can “float” within the tissue. The implant penetrating member(s) 122 may be made of any biocompatible material, such as but not limited to tungsten, silicon and polymers. They can have any tip angle or shape, such as blunt, rounded, or angled. Implants 121 of multiple penetrating members 122 may have any number, distribution and arrangement of electrode shanks within the implant 121. One example is a 2×4 microarray, though other configurations are contemplated. In other embodiments, the implant could be composed of material to transmit light into or from the neural tissue, such as penetrating members containing optical fibers, or of a material to transmit fluid flow into or from the neural tissue, such as penetrating members containing fluid channels or dialysis membranes.

A major contribution to the failure of electrodes over time is believed to be the mismatch in stiffness between the target tissue and the neural implant. For example, a stiff or rigid penetrating member 122 can injure the surrounding softer tissue as a result of mechanical motion, which can in turn induce a damaging tissue response. Flexible and/or ultra-fine implants, such as 7-8 μm diameter carbon fibers, are therefore desirable but are much more difficult to insert through tough tissues, such as peripheral nerve targets. The challenge of inserting electrode penetrating member(s) 122, particularly a flexible ones, is to ensure the force required to penetrate the tough epineurium layer in the peripheral nervous system remains below the buckling force of the implanted penetrating member(s) 122 of the neural implant 121.

The present system 100 includes a vibrational actuator 110, depicted in FIG. 1, configured to generate vibrations or oscillations (which terms may be used interchangeably) to reduce the insertion forces of the penetrating electrode penetrating members 122. It reduces the insertion forces for both “stiff” electrode penetrating members 122 such as microwire or silicon material, as well as “flexible” electrode penetrating members 122 such as comprised of carbon fibers, polyimide or parylene substrates. The vibrational actuator 110 may be an ultrasonic actuator capable of generating vibrations in the ultrasonic range 5-20 μm. It may be operated at a resonant frequency in the range of 20-30 kHz and may preferably be operated at a resonant frequency of about 25 kHz in at least one embodiment. Vibration displacement output may be controlled by increasing and decreasing the driving power provided to the vibrational actuator 110.

The vibrational actuator 110 may be any motor capable of generating vibrations, preferably axial vibrations. For instance, in at least one embodiment the vibrational actuator 110 may be a piezoelectric stack actuator with 25 kHz resonant frequency. In other embodiments, the vibrational actuator 110 may be a voice-coil motor capable of generating vibrations at a lower frequency, such as in the range of about 100-200 Hz, and higher displacements (also referred to as amplitudes) such as up to hundreds of microns. In other embodiments of the system 100, such as may be used in laparoscopic and other applications, the vibrational actuator 110 may be capable of generating vibrations with amplitudes in the range of 0.05 to 0.5 mm and at frequencies in the range of about 80-200 Hz.

The electrode placement system 100 also includes a translational motor 140 interconnected to the implant 121 and capable of moving the implant 121 in a linear fashion into the target neural tissue 5. The translational motor 140 may be any suitable motor, such as but not limited to a linear motor, screw driven motor, conveyor belt, track-based motor, rack and pinion motor, rotational motor, hydraulic motor and others. The translational motor 140 may be configured to advance the implant 121 at suitable velocities, such as in the range of about 0.1 μm/s to 5 mm/s in some embodiments, more preferably in the range of about 0.5 μm/s to 1 mm/s. In at least one embodiment the translational motor 140 may be operated at a speed of about 50 μm/sec. The speed of operation of the translational motor 140 may be set or variable and may be determined by the power supplied to it, such as up to 5 watts in at least one embodiment. The translational motor140 may provide insertion displacements in the range of about 100 μm to 20 cm, and preferably in the range of 100 μm to 10 cm in at least one embodiment.

The electrode placement system 100 also includes a control unit 130, as shown in FIG. 1. The control unit 130 may be separate from the rest of the system 100, such as in tabletop embodiments as shown in FIG. 2, or may be included in the handpiece for hand-held embodiments. Regardless of where positioned, and with reference to FIG. 1, the control unit 130 is in electrical communication with the vibrational actuator 110 and translational motor 140 to provide operative instructions and power to them for operation. The control unit 130 includes a processor 132 that provides instructions for the vibrational actuator 110 and translational motor 140, which may be based on user input to the control unit 130 through an interface (not shown), such as but not limited to LabVIEW-based graphical user interface for control of the vibrational actuator 110 and translational motor 140 with integrated insertion data acquisition. These instructions, which may be provided as electrical impulses such as voltage, are sent from the processor 132 to the vibrational driver 134 and translational driver 136, respectively. The drivers 134, 136 then relay the instructions for activation and deactivation, as well as the various other operative parameters, to the respective vibrational actuator 110 and translational motor 140. For instance, the vibrational driver 134 may send instructions for vibration amplitude, displacement, frequency, power or other parameters, and may drive the vibrational actuator 110 at or near its resonant frequency, depending on the particular vibrational actuator 110 used and the material and/or tissue being penetrated. The translational driver 136 may provide instructions for speed and position of the translational motor 140.

In some embodiments, the electrode placement system 100 may also include a visualization aid 159, as shown in FIGS. 2A and 2B, for viewing and/or visualization of the target insertion site. The lens of the visualization aid 159 may therefore be positioned proximate to the target insertion site to obtain a view of the insertion site. It may also be used to magnify the insertion site for increased accuracy of targeting and placement. In some embodiments, the visualization aid 159 may be a camera, as in FIGS. 2A and 2B. In other embodiments, the visualization aid 159 may be a magnifier. In other embodiments, it may be or include a laser, such as for targeting, and a light source such as to provide additional lighting for better visualization, particularly when the insertion site is deeper in tissue. In certain embodiments, such as laparoscopic applications, the visualization aid 159 may be inserted through a working channel of the laparoscope to the target site, which may be the same or different working channel through which the implant 121 is inserted.

### Target Stabilization

The electrode placement system 100 also includes a target stabilization assembly 150, as depicted in FIG. 1. The target stabilization assembly 150 is configured to hold a portion of the target tissue, such as that surrounding the desired insertion site, steady while the implant 121 is inserted. By temporarily fixing the target insertion site in place, the accuracy of placement is enhanced. Insertion is more reliable and occurs with lower force and less compression of the neural tissue, such as but not limited to dorsal root ganglion, spinal cord, sciatic nerves and pudendal nerve. The present invention contemplates a number of configurations for the target stabilization assembly 150. For instance, in at least one embodiment, the target stabilization assembly 150 may include a stereotaxic frame 151 as shown in FIG. 2A which can be used in tabletop settings, such as for inserting implants 121 into cortical tissue and brain tissue. The stereotaxic frame 151 may include a vertical translation bar 142 and horizontal translation bar 144 to provide adjustments such that the vibrational actuator 110 and attached implant 121 can be driven at any angle in relation to the targeted neural tissue 5. The implant 121 is driven along an insertion axis 128 into the neural tissue 5 at an insertion site. This insertion axis 128 may approach the neural tissue at any angle, such as but not limited to 15°, 45° and 90° relative to the surface of the neural tissue 5. The vertical translation bar 142 may be used to advance the implant 121 into the target site, which may be a distance in the range of up to 50 mm from the initial starting location, preferably up to 10 mm. In other embodiments, the frame 151 may be mounted to and supported on a subject's head, rather than on a table, such as with the placement of deep brain stimulation probes.

The frame 151 may also include a first arm 152 and opposite second arm 154 each mounted to the frame 151 and each terminating in a finger 156, 158, respectively. The arms 152, 154 are selectively moveable in the frame 151 relative to one another to increase and decrease the distance between the respective fingers 156, 158. In at least one embodiment, the arms 152, 154 are selectively movable in a linear direction toward and away from one another. This linear direction may be transverse or orthogonal to the insertion axis 128 of the implant 121 for insertion. The fingers 156, 158 are configured to contact and hold the neural tissue 5 in position, as shown in FIG. 2A, with a detailed view showing arm 154 and finger 158 in FIG. 2B. This may include contact with the ear, mouth, and/or skull to stabilize brain tissue, for instance. When the arms 152, 154 are advanced far enough that the fingers 156, 168 are sufficiently gripping the sides of the tissue or skull, the locking mechanisms 153, 155 can be activated to secure the arms 152, 154 in position, respectively, as shown in FIG. 2A. The locking mechanisms 153, 155 may be pins, switches, knobs, screws, or other similar component providing enough frictional force on the arms 152, 154 to prevent them from further movement. Once the implant 121 is inserted as desired, the grip on the skull or other surrounding tissue may be released by disengaging the locking mechanisms 153, 155 and moving the arms 152, 154 in the opposite direction away from one another to disengage the fingers 156, 158 from the tissue 5.

In some embodiment, the target stabilization assembly 150 may be sufficiently elongate, narrow and flexible to be inserted through a channel of a laparoscope. In such embodiments, target stabilization assembly 150 and insertion assembly 120 discussed below must be able to work at some considerable distance from the operator, possibly after passing through a trocar, and occasionally under significant curvature before reaching the nerve target. Therefore, in such embodiments, the target stabilization assembly 150, insertion assembly 120 and implant 121 may be compatible in size with minimally invasive surgical approach, such as through a 5 mm laparoscopic port or trocar, and where the target neural tissue may be at a distance of about 10 cm and have a diameter of less than 4 mm.

The target stabilization assembly 150, insertion assembly 120 and implant 121 may therefore be included or housed in a delivery stem having an inner core and surrounding outer sheath. The inner core may be a guide wire or other similar elongate structure that is sufficiently flexible to pass through the curvature necessary for a laparoscopic approach but also rigid enough to provide structural support and transmit vibrations from the vibrational actuator 110 located outside of the laparoscope to the implant 121 at the distal end of the inner core. Accordingly, the inner core may be the insertion assembly 120 in laparoscopic embodiments. The outer sheath may be a semi-flexible, low friction material such as Teflon or nylon that surrounds the inner core and enables the delivery stern to be gripped from the outside without significantly damping the oscillation of the inner core. The outer sheath may be retracted for insertion of implant. An endoscopic-style manipulator may also be inserted with flexibility, but then made rigid with a cabling system. A visualization aid 159, such as a camera and/or light, may also be inserted through a channel of the laparoscope.

In some applications, such as penetration of peripheral targets like dorsal root ganglion (DRG) and peripheral nerves, insertion is more challenging because the targets are tougher and have increased freedom of movement. In addition, for the peripheral nervous system, anatomy is often more variable between subjects and stereotaxic approaches are far less useful and common. Therefore, surgical approaches for electrode placement may be more reliant on manual, handheld equipment as there is often not a good way to mount hardware or fixturing.

Accordingly, in certain embodiments of the electrode insertion system 100, the target stabilization assembly 150′ may be handheld, as shown in FIGS. 3-11. With particular reference to FIGS. 3-5B, the target stabilization assembly 150′ may include a housing 151′ that replaces the frame 151 discussed above. This housing 151′ may include the vibrational actuator 110 and translational actuator 140, as well as the insertion assembly 120 which allows positioning, placement, advancement and vibration of the electrode penetrating member(s) 122 as discussed above.

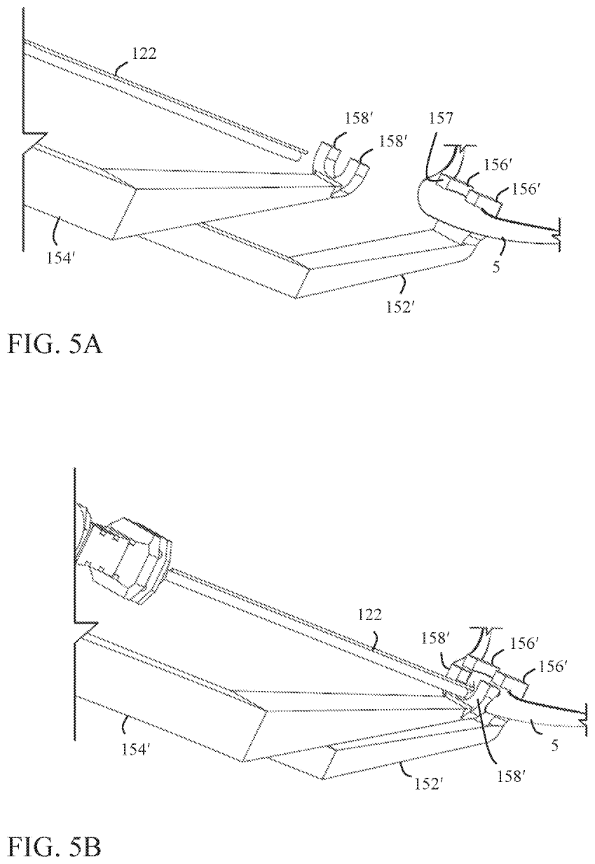

In this embodiment, the target stabilization assembly 150′ includes at least one arm 152′, and preferably includes a first arm 152′ and second arm 154′ each having an elongate length extending from the housing 151′ parallel to the insertion axis 128 for the electrode penetrating member(s) 122 and/or implant 121. Each arm 152′, 154′ includes at least one finger 156′, 158′, respectively, and preferably has more than one finger 156′, 158′. In at least one embodiment, as shown in FIGS. 3-5B, the fingers 156′, 158′ may be located at the terminal ends of the elongate arms 152′, 154′. The fingers 156′, 158′ are dimensioned and configured to engage the neural tissue 5 such as by gripping or holding the tissue 5 relative to the surrounding tissue. In the embodiments shown in FIGS. 3-6, the fingers 156′, 158′ may extend transversely, or at least partially transversely, away from the length of the respective arm 152′, 154′. There may be multiple fingers 156′, 158′ on each arm 152′, 154′, such as at least one but preferably two or three. In many embodiments, the fingers 156′, 158′ may form a space between them, as shown in FIGS. 3-6, sufficiently large enough for the electrode penetrating member(s) 122 and/or entire implant 121 to pass through to reach the neural tissue 5 held therein. The arms 152′, 154′ and fingers 156′, 158′ may therefore be positioned so the space between fingers 156′, 158′ is aligned with the insertion axis 128. In some embodiments, the distal or first arm 152′ may have a single finger 156′ that may be solid and lacking any space or opening. It may therefore provide a backstop for the neural tissue 5 to better stabilize it. In other embodiments, tissue stabilization may be better achieved with multiple fingers 156′ on the first or distal arm 152′, as shown in FIGS. 3-5B. The fingers 156′, 158′ may also be the same or different from one another in the same set or as between the different sets. For instance, in some embodiments the fingers 156′ on the first arm 152′ may further include extensions 157 that extends from the first fingers 156′ on the first arm 152′ toward the fingers 158′ of the second arm 154′ and are dimensioned to fit over the corresponding second fingers 158′ of the second arm 154′, as shown in FIGS. 3 and 5A-5B. The extensions 157 may also fit over the neural tissue 5 when positioned between the first and second fingers 156′, 158′, as shown in FIG. 5B, to encase the neural tissue 5 and further stabilize it for penetration.

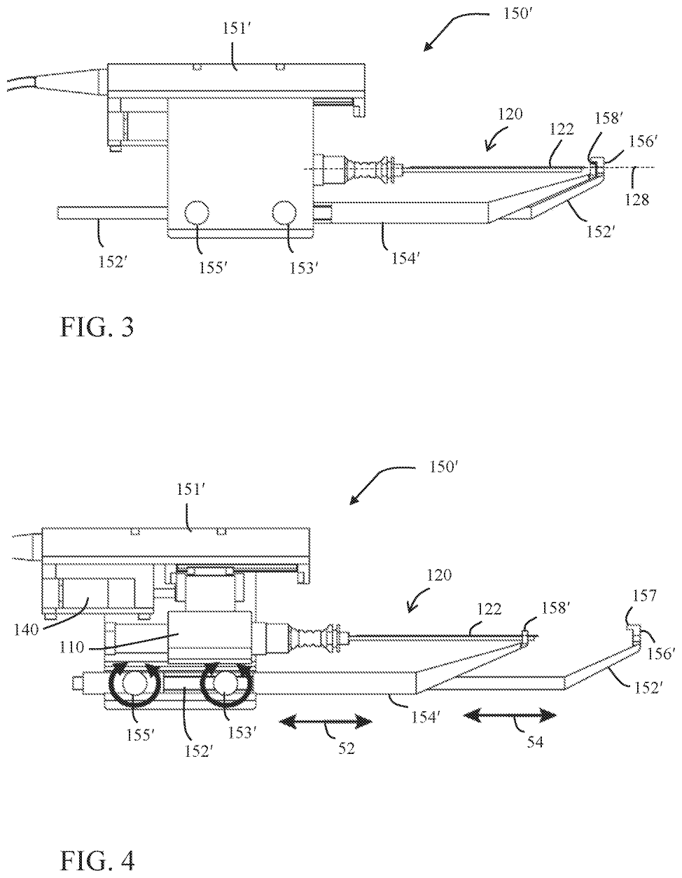

Each arm 152′, 154′ is also selectively moveable relative to one another and relative to target neural tissue 5 to grasp and secure the target neural tissue 5 in a fixed or stationary position relative to the electrode penetrating member 122 and/or implant 121 for more precise insertion. Not only is the target stabilization assembly 150′ handheld and moveable in space by the user, the first and second arms 152′, 154′ are also selectively moveable in a longitudinal axis that is parallel to the insertion axis 128 of the insertion assembly 120. For instance, as shown in FIG. 4, the first arm 152′ may be movable in the longitudinal direction shown by arrow 52, and the second arm 154′ may be movable in the same longitudinal direction as indicated by arrow 54. In at least one embodiment, each arm 152′, 154′ is moveable. In other embodiments, at least one of the arms 152′, 154′ may be moveable, which may be either the first or second arm 152′, 154′. The arms 152′, 154′ may be positioned adjacent to one another, such as side by side, or in a preferred embodiment may be positioned co-axially such that one is inserted and moveable within the other. For instance, as shown in FIGS. 3-5B, the first arm 152′ being the more distally located arm may be received and movable within the second arm 154′ being the more proximally located arm In other embodiments, however, this may be reversed. The arms 152′, 154′ may have a polarized configuration that prevents rotational movement relative to one another in order to maintain their alignment. For instance, in at least one embodiment as shown in FIGS. 3-5B, the arms 152′, 154′ may have a square cross-section. In other embodiments, they may have a triangular cross-section or other angled cross-section that limits, inhibits or prevents rotational motion. In still further embodiments, the arms 152′, 154′ may have a cylindrical cross-section to allow them to be rotated about their own respective axis, which may also be parallel to the insertion axis 128, to allow the fingers 156′, 158′ to be rotated away or toward the neural tissue 5, such as to come behind tissue.

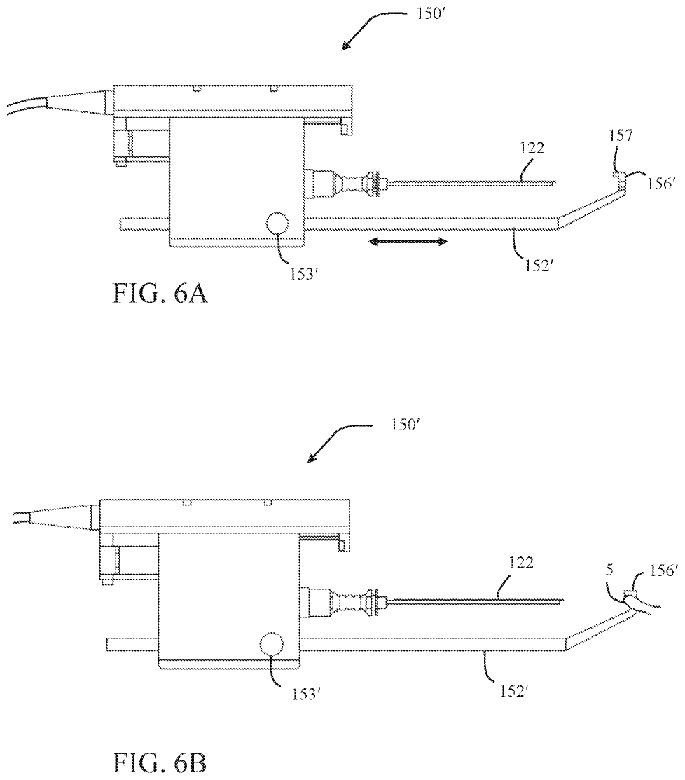

The target stabilization assembly 150′ also includes at least one locking mechanism 153′, as shown in FIGS. 6A and 6B, or multiple locking mechanisms 153′, 155′ as shown in FIG. 4. The locking mechanism 153′ is configured to secure an arm 152′ in a particular position to keep it from moving further in the longitudinal direction. For instance, there may be 156′, 158′ a first locking mechanism 153′ configured to secure the first arm 152′ in position and a second locking mechanism 155′ configured to secure the second arm 154′ in position. In at least one embodiment, the locking mechanisms 153′, 155′ may be screws, set screws, bolts, wingnuts or other such device that may extend through the exterior of the housing 151′ at one end and may terminate against a portion of the respective arm 152′, 154′. When rotated in one direction to tighten down, the locking mechanism 153′, 155′ may increase the friction between the arms 152′, 154′ themselves and/or with the housing 151′ or an internal part of the housing to retain the arms 152′, 154′ in position. When rotated in the opposite direction, the locking mechanisms 153′, 155′ may loosen the frictional fit against the arms 152′, 154′ so they may be moved in the longitudinal direction. To move, each arm 152′, 154′ may each be gripped and manually moved individually by a user of the system 100 then locked in place once the desired position is achieved. Though described as rotating to secure and loosen hold on the arms 152′, 154′, in other embodiments the locking mechanisms 153′, 155′ may act by tightening and loosening on the arms 152′, 154′ by mechanisms other than rotation, such as but not limited to gripping, compression, ratcheting and spring-biased compression.

In some embodiments, each arm 152′, 154′ may be affixed and slidable along its own track. The locking mechanism 153′, 155′ may extend through arm 152′, 154′ and may be tightened down onto the track when the appropriate location along the track is achieved, In some instances, the locking mechanism 153′, 155′ may be affixed to the respective arm152′, 154′ and the portion of the locking mechanism 153′, 155′ that extends through the housing 151′ may be used as a handle to move the arm 152′, 154′, such as by sliding, in the longitudinal direction to adjust the position and achieve the desired location, then may be turned to secure the arm 152′, 154′ in place. In such embodiments, the housing 151′ may include an aperture through which a portion of the locking mechanism 153′, 155′ may extend and may be movable within. In such embodiments, the aperture of the housing 151′ may therefore limit the degree of movement of the arms 152′, 154′.

In action, at least one of the arms 152′, 154′ may be extended from the housing 151′ in a longitudinal direction until the fingers 156′, 158′ are positioned on either side of a target neural tissue 5, as shown in FIG. 5A, such as with one set of fingers 156′ on the distal side of the target tissue 5 and another set of fingers 158′ positioned on the proximal side of the target tissue 5 relative to the electrode penetrating member(s) 122 and/or implant 121. The target tissue 5 may then be secured and/or stabilized by moving the arms 152′, 154′ toward one another, or by moving one of the arms 152′, 154′ toward the other while the other remains stationary, until the target tissue 5 is retained between the fingers 156′, 158′, as shown in FIG. 5B. In embodiments having only a single arm 153′, as in FIGS. 6A and 6B, the arm 152′ may be extended and the fingers 156′ positioned distally behind the target neural tissue 5, as shown in FIG. 6B. Once so positioned, the arm 153′ may be locked in place with the fingers 156′ holding the target tissue 5, or the arm 152′ may be moved slightly in the proximal direction to apply slight tension to the target tissue 5 to better secure it in place. ‘The arm 152’ may then be secured in place.

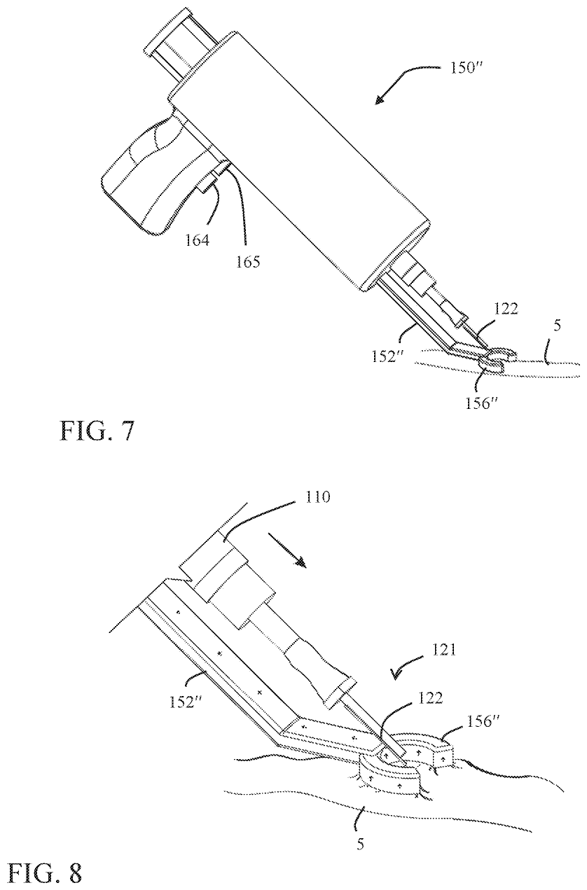

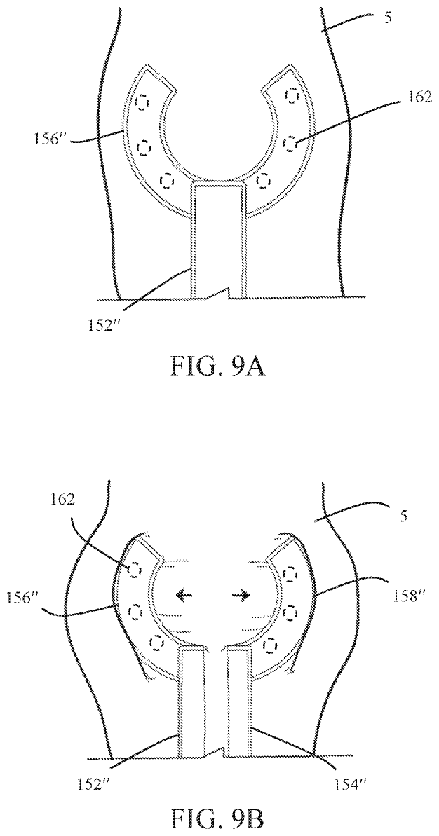

In still further embodiments, the target stabilization assembly 150 may utilize vacuum to hold the target neural tissue 5 in position. For instance, as shown in FIGS. 7-9A, the target stabilization assembly 150″ may include an arm 152″ that extends to the target insertion site and includes a channel therein through which a negative pressure is applied. The handpiece housing 151″ may include a vacuum control 164 button to turn the vacuum on and off, and an insertion control 165 to control the advancement and retraction of the implant 121. The distal end of the vacuum arm 152″ may include at least one finger 156″ also having a channel in fluid communication with the channel of the vacuum arm 152″ through which the negative pressure may be applied. The finger 156″ includes at least one vacuum port 162 (not shown) through which the vacuum may be drawn. The finger 156″may be positioned along the surface of the neural tissue 5, such as epineurium of a nerve fiber or bundle, with the vacuum port(s) 162 adjacent and/or abutting the neural tissue 5. When the vacuum control 164 is activated, the negative pressure draws the surface of the neural tissue 5 into contact with finger 156″, as shown in FIG. 8. As long as the vacuum is maintained, the neural tissue 5 is held in place against the finger 156″. The implant 121 may then be vibrated and inserted. As shown in FIGS. 7-9A, the finger 156″ and vacuum ports 162 may at least partially surround or encircle the insertion site of the neural tissue 5. This placement provides a taut surface for insertion to limit perturbation and increase accuracy. In some embodiments, there may be multiple fingers 156″, 158″,as shown in FIG. 9B, which may be selectively moveable relative to one another to increase the distance between the fingers 156″, 158″. This further increases the tension on the neural tissue 5 surface for further reduction in surface movement during insertion. In such embodiments, each finger 156″, 158″ includes a channel in fluid communication with the channel of the vacuum arm 152″ and/or each finger 156″, 158″ corresponds to a respective vacuum arm 152″, 154″ each providing negative pressure to the respective fingers 156″, 158″ before, during and/or after spreading.

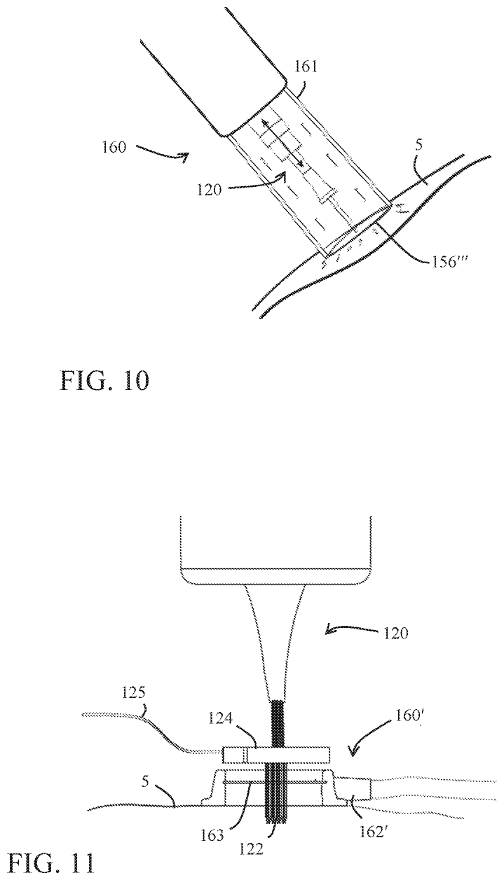

In still further embodiments, as shown in FIG. 10, the target stabilization assembly 150 includes a basket 160 which may have a conical, ring or similar shape and is positionable entirely surrounding and/or encircling the target insertion site with the application of vacuum. The basket 160 may include a number of stiffening supports 161 and at least one finger 156′″ that may be a ring positionable in contact with the surface of the neural tissue 5. A vacuum may be drawn in the inner volume of the basket 160 formed between the ring finger 156′″ and the stiffening supports 161. This may be used in connection with the outer sheath of a laparoscopic embodiment, for example.

In a still further embodiment, as shown in FIG. 11, the target stabilization assembly 150 may include a low-profile button 160′ that is positionable in contact with the surface of the neural tissue 5. The low-profile button 160′ may include a membrane seal 163 spaced apart from the surface of the neural tissue 5 and a vacuum port 162′ located therebetween. A negative pressure may be drawn through the vacuum port 162′creating a vacuum between the membrane seal 163 and the surface of the tissue. The electrode penetrating member(s) 122 of the implant 121 may be inserted through the membrane seal 163 while vacuum is applied. The membrane seal 163 may be made of a self-sealing material, such as but not limited to silicone rubber or polyurethane such that the vacuum remains in place even when the electrode penetrating member(s) 122 are inserted. Since the electrode penetrating member(s) 122 of the implant 121 are inserted through the low-profile button 160′ into the neural tissue 5, the low-profile button 160′ may remain in place on the neural tissue 5 for the duration of use of the implant 121, such as for stimulation of the tissue and/or collecting data from the tissue.

### Implant Stabilization

Keeping the implant 121 stabilized during insertion is important for accuracy and precision of placement at the desired target site. This is true, and can be challenging, when the implant 121 is a single electrode penetrating member 122 but is particularly challenging for implants 121 having multiple electrode penetrating members122, such as floating arrays which are not fixed to bone once implanted and may therefore designed to “float” or move freely with the neural tissue, and for microelectrodes having an extremely small scale requiring heightened precision. The need for implant 121 stabilization is even more important in view of the vibrations that are transmitted to the implant 121 during insertion from the vibrational actuator 110. Indeed, floating arrays typically have a highly flexible cable of wires, called a tether or cable 125, connected to a base 124 of the implant 121, as depicted in FIGS. 13A and 1313, that provides electrical communication between the electrodes located on the penetrating members 122. of the implant 121 and the controller 130 which may send and receive information to and from the electrode penetrating members 122 to send and receive real time electrical signals while the implant 121 is being inserted, providing a more accurate placement of the implant 121 to the target depth. The cable 125 may include a single channel of electrical communication or multiple channels for the same or different types of communication. For example, the cable 125 may include multiple channels each dedicated and connected to a single electrode from the array 121. In other embodiments, the cable 125 may include multiple channels, such as at least one providing electrical communication with electrodes and at least one other channel providing light energy such as in the case of use of a fiber optic to deliver and/or collect light and light-based data. In still other embodiments, the various channels of the cable 125 may be configured and dimensioned to enable the transmission of fluid flow into or from the neural tissue, such as in the case of a probe or cannula. This flexible cable 125 is also vibrated with the implant 121 as a result of being connected thereto and adds additional strain to the implant 121 that can further impair accurate placement.

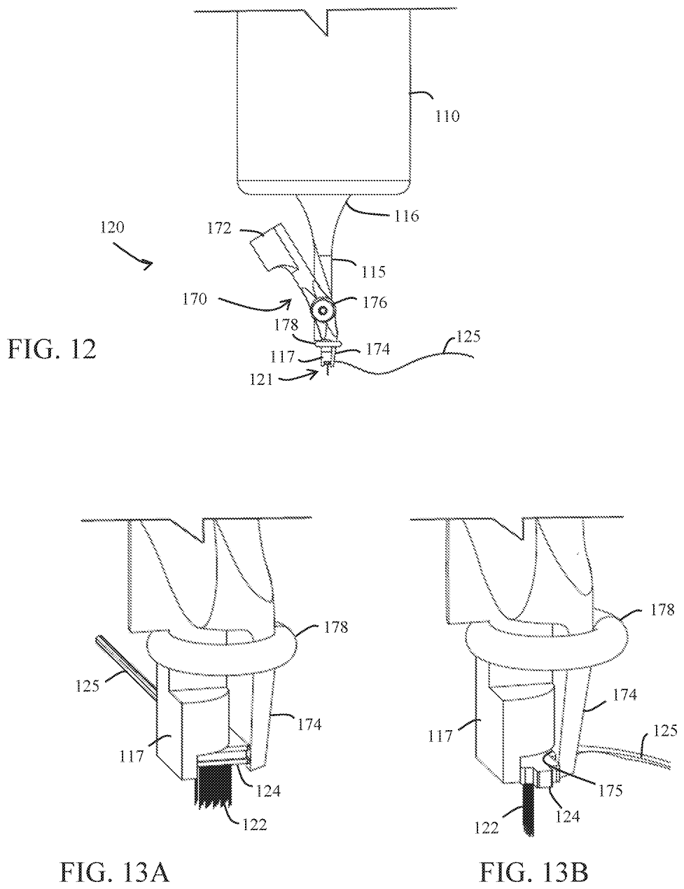

To overcome this, the electrode placement system 100 of the present invention includes an insertion assembly 120 configured to secure the implant 121 during vibration and insertion, and then selectively release the implant 121 once inserted into the desired target site without perturbing the final placement of the implant 121 in the insertion site. As shown in FIGS. 12-14D, the insertion assembly 120 provides a secure attachment of the implant 121 for insertion and faithful transmission of the vibrations from the vibrational actuator 110, yet quick release when desired without perturbing or disrupting the insertion site. Specifically, and with reference to FIG. 12, the insertion assembly 120 includes a horn 115 having a horn base 116 secured to and extending from the vibrational actuator 110 at one end, and a horn tip 117 at the opposite end. The horn 115 is made of a rigid material suitable for transmitting vibrations therethrough, such as but not limited to titanium. In at least one embodiment the horn base 116 is a continuous piece threading into the tail mass of the vibrational actuator 110. For instance, the vibrational actuator 110 may be a piezoelectric motor having a piezoelectric stack which may be positioned between the horn base 116 and the tail mass of the piezoelectric motor. The horn 115 may preferably have a narrowing diameter from the horn base 116 to the horn tip 117, with the horn tip 117 having a smaller diameter than the horn base 116. In some embodiments the horn tip 117 may be as small as a 30 G hypodermic needle. This smaller diameter allows the horn tip 117 to fit into small surgical openings of a size of 1 mm, allowing the system 100 to be used for placement of implants 121 on the order of 10s to 100s of microns.

The horn tip 117 is dimensioned to abut at least a portion of the implant 121 therein, such as the base 124 of an implant 121 as shown in FIGS. 12-14D. The implant 121 may be any type having at least one electrode penetrating member122 mounted to and extending away from a base 124. For instance, the implant 121 may be a floating array or microarray, such as but not limited to Blackrock Utah array and Neuronexus Matrix array, having two, three, four, six, eight, twelve, or more electrode penetrating members 122. The base 124 may hold all the electrode penetrating members 122 together in the implant 121. A cable 125 may also connect to the base 124 in electrical communication with the electrode sites located in the penetrating members 122, such as one or more conductors in the cable 125 to each electrode site on the penetrating member(s) 122 of the implant 121. In other embodiments, the implant 121 may be a single penetrating member 122, which may be mounted to and extend from a post as a base 124. A cable 125 may also connect to single or multiple electrode sites on the electrode penetrating member 122, or a single wire may connect thereto.

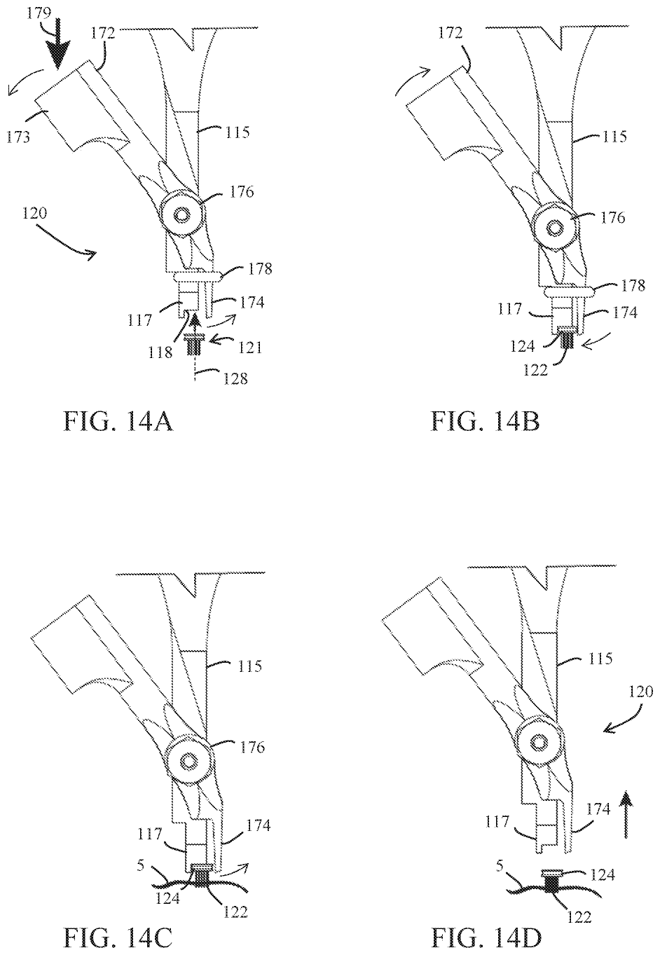

The horn tip 117 is configured to support a portion of the base 124 of the implant 121. For instance, in some embodiments the base 124 of the implant 121 may be held against the horn tip 117 by the implant stabilizer 170, described below in greater detail. In at least one embodiment as shown in FIGS. 13A-14A, the horn tip 117 may include a recess 118 correspondingly dimensioned to the size and shape of at least a portion of the base 124, which may include the entire base 124 of the implant 121. This recess 118 is therefore configured to receive the implant 121, such as the correspondingly dimensioned base 124 therein, when loading the implant 121 into the insertion assembly 120 as depicted in FIG. 14A. The recess 118 may be shaped as a notch, groove, angled or curved pocket or have other configuration suitable to provide further stability to the implant 121 during insertion. Preferably, the shape and dimensions of the recess 118 are sufficient to limit or prevent rotational movement of the implant 121 during insertion or placement into the target tissue. In at least one embodiment, the horn tip 117 does not contact the penetrating member(s) 122 extending from the base 124 even when the implant 121 is fully seated within the recess 118 of the horn tip 117.

The insertion assembly 120 also includes an implant stabilizer 170 configured to securely hold the implant 121 against the horn 115 with a sufficient force to allow the transmission of vibrations from the vibrational actuator 110 through the implant 121 to the penetrating members 122 during insertion, but also is selectively releasable from the implant 121 without perturbing the placement or position of the implant 121 once inserted. The implant stabilizer 170 includes a first end 174 positioned proximate to the horn tip 117, and therefore implant 121. The first end 174 of the implant stabilizer 170 is capable of selectively contacting a portion of the implant 121, such as the base 124, to hold the implant 121 in place against the horn tip 117. The point of contact between the first end 174 and the implant 121 should be as close to the penetrating members 122 as possible without interfering with them or the vibration transmitted to them, to provide the most control over the implant 121 during insertion. In at least one embodiment, the first end 174 may have a solid construction, as shown in FIG. 13A, which may provide additional support to the implant base 124 and may be useful when the implant 121 is mounted in the insertion assembly 120 with the penetrating members 122 laterally positioned so the cable 125 extends away from the implant 121 and does not impede the first end 174. In other embodiments, as in FIG. 13B, the first end 174 may include an aperture 175 sufficiently sized and dimensioned to permit the passage of the array cable 125 therethrough, such as when the array 121 is mounted within the insertion assembly 120 so the electrode penetrating members 122 are positioned longitudinally and the first end 174 would otherwise clamp down on the cable 125. This aperture 175 may be located at a terminal end of the first end 174 or anywhere along the length of the first end 174. The first end 174 is made of material that is sufficiently sturdy that even with an aperture 175, the first end 174 is capable of providing sufficient force to the base 124 of the implant 121 to hold it in place against the horn tip 117 and/or recess 118 therein.

The implant stabilizer 170 also includes a second end 172. opposite from the first end 174. In at least one embodiment, the second end 172 and first end 174 of the implant stabilizer 170 may be of a unitary construction, though in certain embodiments they may be separate pieces joined together. The implant stabilizer 170 is displaceably mounted to the horn 115 such that it is secured to the horn 115 at a connection point 176 and yet is also bendable about the connection point 176 upon the application of force. Each of the second end 172 and first end 174 may have an elongate length to enable torque to be provided through the connection point 176. The implant stabilizer 170 may be made of material such as acetal, though it may be made of any stiff yet bendable or resilient material such as plastics, polymers, polymer blends, rubber, and other natural or synthetic materials such as having a hardness of greater than 60A Shore, though more optimally greater than 70D Shore. The second end 172 may extend away from the connection point 176 in the direction of the horn base 116. In at least one embodiment, the second end 172 may extend at an angle relative to the insertion axis 128 such that it deviates from the axis. The first end 174 may also extend at an angle relative to the insertion axis 128, which may be the same or different angle relative to the insertion axis 128 as that of the second end 172. In at least one embodiment the angle the first end 174 deviates from the insertion axis 128 is less than that of the second end 172. The angles of the first and second ends 174, 172 relative to the insertion axis 128 may be any angle up to 140 degrees. For instance, in at least one embodiment the first end 174 is positionable at an included angle in the range of 10-60 degrees relative to the insertion axis 128, depending on whether force is applied or not. Similarly, the second end 172 may be positionable at an included angle in the range of 25-90 degrees relative to the insertion axis 128, depending on whether force is applied or not.

The second end 172 may also have a tab 173 that protrudes from the second end 172. The tab 173 may extend or protrude from any location along the second end 172, such as at a terminal end, or along the length of the second end 172 such as a fin and may have any shape or configuration. In at least one embodiment, the tab 173 may have a generally square or rectangular shape, as shown in FIGS. 12 and 14A-14D, though it may also have a triangular, curved or non-linear shape. The tab 173 has a shape and extends by a length from the second end 172 sufficient to enable gripping by surgical tweezers or other similar gripping mechanism that can be operated by a user proximally for distal action at or within the surgical or insertion site, which may be implemented in surgical openings or through the working channel of a laparoscope for minimally invasive applications.

The implant stabilizer 170 secures to the horn 115 at the connection point 176, which may be at any location along the length of the horn 115 such as along the insertion axis 128 in some embodiments and off-axis from the insertion axis 128 in other embodiments. In at least one embodiment the connection point 176 attaches the implant stabilizer 170 to the horn 115 at a position that aligns the terminal end of the first end 174 with terminal end of the horn tip 117, such as with the recess 118 formed therein. This positioning allows the maximum torque to be applied by the first end 174 on the implant 121 when held in place. However, in other embodiments the connection point 176 may attach lower on the horn 115, closer to the horn tip 117, which may provide increased control of the implant 121 or may allow the first end 174 to extend beyond the base 124 of the implant 121 when holding the base 124 of the implant 121 and/or the horn tip 117. The connection point 176 may include hardware for connection, such as a screw, nut, bolt, washer, bearing and combinations thereof, and may include welding or adhesive in certain embodiments. Regardless of the material used for the connection point 176, it is preferably made of a material that is sufficiently light that it does not dampen or otherwise interfere with the vibrations from the vibrational actuator 110. Examples may include, but are not limited to, stainless steel, nylon, or polyether ether ketone (PEEK).

The implant stabilizer 170 has a construction and is of a material that permits movement of the second end 172 about the connection point 176 when force is applied to the second end 172. Specifically, force may be applied to the second end 172 in the direction of arrow 179, shown in FIG. 14A, which temporarily moves the second end 172 to further deviate from the insertion axis 128. When force is applied to the second end 172, it may be at an angle of 45°-90° relative to the insertion axis. When force is no longer applied to the second end 172, as in FIG. 14B, the resilient nature of the material combined with the secure attachment at the connection point 176 results in the second end 172 moving in the direction of the insertion axis 128 back to its relaxed position, which is still deviated or at an angle relative to the insertion axis 128, such as in the range of about 30°-45°. The force on the second end 172 required to move it may be a pushing force applied to the proximal end of the second end 172, as shown in FIG. 14A, or it may be a pulling force applied to the tab 173. the forces applied to the second end 172 of the implant stabilizer 170 could be generated by a user's fingers in at least one embodiment, or through mechanical means such as through a spring, level, and/or ratcheting mechanism which is partially mounted to the housing of the vibrational actuator 110 in other embodiments.

In at least one embodiment, the insertion assembly 120 also includes a biasing member 178 contacting the first end 174 and providing a biasing force such as torque against the first end 174 to hold the first end 174 against the horn 115, and therefore against the implant 121. As shown in the embodiment of FIGS. 12-14C, the biasing member 178 may be an O-ting in some embodiments that surrounds or at least partially surrounds the first end 174 and the horn tip 117, though in other embodiments the biasing member 178 may not be an O-ring. The biasing member 178 contacts both the first end 174 and the horn tip 117, such as outer-facing sides of each, in order to urge the first end 174 in the direction toward the horn tip 117, Despite this contact, the biasing member 178 also permits some limited movement of the first end 174 away from the horn tip 117, such as when force is applied to the first end 174 through the connection point 176 from the second end 172. Under such circumstances, biasing member 178 permits the first end 174 to flex to an angle of about 15° relative to the horn tip 117 and/or insertion axis 128 when force is applied to the second end 172, When no force is applied, however, the biasing characteristics and positioning of the biasing member 178 urges the first end 174 in the direction of the horn tip 117. Under the biasing force of the biasing member 178, the first end 174 is positioned at an angle of about 0-5° relative to the horn tip 117 and/or insertion axis 128. The biasing member 178 may achieve this in part as a result of its material construction, which may be of resilient plastics, polymers and rubbers such as Buna-N or other nitrile based rubbers, and may be synthetic or naturally derived. In other embodiments, the biasing member 178 may be a spring, a ratchet mechanism, hydraulic, vacuum, or electromagnetic mechanism configured to apply torque or other force to the first end 174 in the direction of the horn tip 117. In some embodiments, the biasing member 178 may also be selectively released from the insertion assembly 120, such as by cutting or snipping, to disengage it from the first end 174. This causes the first end 174 to return to its relaxed, unbiased position and therefore to move away from the horn tip 117 and/or to otherwise release its grip on the implant 121.

FIGS. 14A-14D demonstrate the use of the insertion assembly 120 through one embodiment thereof As shown in FIG. 14A, an implant 121 may be loaded into the insertion assembly 120 by applying force in the direction of arrow 179 to the second end 172. This force makes the implant stabilizer 170 bend about the connection point 176, in turn causing the first end 174 to rotate away from the horn tip 117. The first end 174 presses and strains against the biasing member 178 as a result of this force, and the biasing member 178 limits the degree of movement of the first end 174 away from the horn tip 117. The recess 118 formed in the horn tip 117 is now unobscured and the implant 121 may be moved into the recess 118 until the base 124 thereof is seated in the recess 118 of the horn tip 117.

Once the implant 121 is fully seated, the force on the second end 172 is relieved and the second end 172 rotates back to its natural position, as shown in FIG. 14B. This also releases the pressure on the first end 174, allowing the biasing member 178 to exert a biasing force on the first end 174 to urge it toward the implant 121 and horn tip 117 until the first end 174 is securely engaging the base 124 of the implant 121 and holding it against the horn tip 117. The system 100 may then be used to vibrate the electrode penetrating member(s) 122 of the implant 121 and drive the implant along the insertion axis 128 to penetrate the neural tissue 5 at the target site as previously described.

Once the electrode penetrating member(s) 122 of the implant 121 are embedded in the target site of the neural tissue 5, the implant stabilizer 170 may be removed from contact with the implant 121 by again applying force to the second end 172, or by selectively releasing the biasing member 178 from the insertion assembly 120, as shown in FIG. 14C, such as by cutting or snipping. Micro-scissors or other surgical cutting instruments may he used to accomplish this, including laparoscopic devices. Once the biasing member 178 is released, the biasing force is no longer applied to the first end 174 and it returns to its natural, unbiased position, rotating slightly about the connection point 176. This releases the gripping engagement on the implant 121, such as the base 124.

Once the biasing member 178 is released and the first end 174 is no longer holding the implant 121, the insertion assembly 120 and rest of the system 100 may be removed from the insertion site, leaving the implant 121 embedded in the neural tissue 5, as shown in FIG. 14D. Accordingly, the retraction of the system 100 is accomplished without disturbing the implant 121 once embedded.

In other embodiments, the implant stabilizer 170 may be a clamp mechanism where the first end 174 is a base and the second end 172 is a top that may be secured to the base such as with screws, such as shown in FIG. 2B. The base is configured to receive and support the implant 121 and the top encloses at least a portion of the implant 121. Once secured with screws, the clamp mechanism securely retains the implant 121 therein and effectively transfers the vibrational energy from the actuator 110 to the implant 121. The clamp mechanism implant stabilizer 170 may connect to the actuator 110 through a Luer connector, which may be a Luer-Lock, Luer-Slip, and combinations thereof, to permit the transmission of vibrations from the actuator 110 to the clamp mechanism implant stabilizer 170. A Luer-Lock provides a fixed orientation for the implant 121, whereas a Luer-Slip allows for the implant 121 to be oriented in a full 360-degree rotation in relation to the target site. This provides the user flexibility when determining the optimal location and/or orientation to insert the implant 121.

In still other embodiments, the implant stabilizer 170 may be a cam positioned adjacent to the implant 121 and having a cam profile that contacts and provides adequate force to the implant 121 when the cam is rotated to allow effective vibrational energy transfer from the actuator 110 to the implant 121 while at the same time not overloading the implant 121 to the point of damaging the electrode penetrating member(s) 122.

In still further embodiments, the implant stabilizer 170 may be polyethylene glycol (PEG) that is potted around the implant 121, such as the base 124 and possibly even portions of the electrode penetrating member(s) 122, to provide structural stability during insertion while also permitting transmission of vibrational energy from the actuator 110 to the electrode penetrating member(s) 122. Once the implant 121 is inserted and embedded in the target neural tissue 5, the PEG may be dissolved by the application of an aqueous solution such as saline, and/or by heating a small wire integrated into the implant 121, such as the base 124 of the implant 121, to facilitate the melting of the PEG following implantation.

Since many modifications, variations and changes in detail can be made to the described preferred embodiments, it is intended that all matters in the foregoing description and shown in the accompanying drawings be interpreted as illustrative and not in a limiting sense. Thus, the scope of the invention should be determined by the appended claims and their legal equivalents. Now that the invention has been described.

What is claimed is:

1. An insertion assembly for inserting a neural implant into target neural tissue, said insertion assembly comprising:
a horn extending between a horn base secured to a vibrational actuator at a first end and a horn tip at an opposite end, said horn tip configured to abut a portion of a neural implant, said horn configured to transmit vibrations generated by said vibrational actuator to said neural implant during insertion; and
an implant stabilizer displaceably mounted on and selectively moveable with respect to said horn, said implant stabilizer having a first end and opposite second end, said first end positioned proximate to and biased to urge against said horn tip to selectively secure said neural implant against said horn tip by selective displacement of said second end, and said first end having a range of motion limited by a corresponding range of motion of and application of force to said second end.

2. The insertion assembly of claim 1, wherein said horn tip further comprises a recess correspondingly shaped to a portion of said neural implant, said recess configured to receive said portion of said neural implant.

3. The insertion assembly of claim 2, wherein said neural implant includes a base and at least one penetrating member extending from said base, and said recess is correspondingly shaped and configured to receive said base of neural implant.

4. The insertion assembly of claim 1, wherein said neural implant has a base, at least one penetrating member extending from said base and having at least one electrode, and a cable in electrical communication with said at least one electrode, said first end of said implant stabilizer having an aperture dimensioned to receive said cable.

5. The insertion assembly of claim 1, wherein said neural implant is a floating array.

6. The insertion assembly of claim 1, wherein said first end of said implant stabilizeris selectively displaceable in an opposite direction to said second end relative to said horn upon said application of force to said second end.

7. The insertion assembly of claim 1, wherein said implant stabilizer is displaceably mounted to said horn at a connection point and said first and second ends of said implant stabilizer are selectively movable relative to said horn by rotation about said connection point.

8. The insertion assembly of claim 7, wherein said first end of said implant stabilizer is rotatable about said connection point in a direction away from said horn tip to release said neural implant by movement of said second end.

9. The insertion assembly of claim 1, wherein said biasing member is resiliently deformable upon said application of force to said second end of said implant stabilizer and is configured to permit said first end of said implant stabilizer to release said neural implant from said horn tip upon said application of force to said second end without perturbing said neural implant once inserted in tissue.

10. The insertion assembly of claim 1, wherein said biasing member is selectively removable from said insertion assembly without perturbing said neural implant once inserted in tissue.

11. The insertion assembly of claim 10, wherein said biasing member is selectively removable by cutting said biasing member.

12. The insertion assembly of claim 1, wherein said horn extends along an insertion axis, said implant stabilizer is mounted to said horn with said first and second ends each positioned at an angle relative to said insertion axis, and said application of force to said second end increases said. angle of said first and second ends relative to said insertion axis.

13. The insertion assembly of claim 12, wherein said first and second ends are positioned in opposite directions from said insertion axis.

14. The insertion assembly of claim 12, wherein said angle of each of said first and second ends relative to said insertion axis is up to 140 degrees.

15. The insertion assembly of claim 12, wherein said angle of said first end relative to said insertion axis is different than said angle of said second end relative to said insertion axis.

16. The insertion assembly of claim 15, wherein said angle of said first end relative to said insertion axis is less than said angle of said second end relative to said insertion axis.

17. The insertion assembly of claim 1, wherein said implant stabilizer allows transmission of said vibrations to said neural implant.

18. The insertion assembly of claim 1, further comprising a biasing member contacting said horn tip and said first end of said implant stabilizer, said biasing member configured to provide a biasing force on said first end of said implant stabilizer toward said horn tip sufficient to secure said neural implant against said horn tip.

19. The insertion assembly of claim 18, wherein said biasing member is configured to allow limited movement of said first member of said implant stabilizer away from said horn tip upon said application of force to said second end.

20. An electrode placement system, comprising:
the insertion assembly as recited in claim 1;
a control unit having a processor, a vibrational driver configured to provide operative vibrational instructions dictating vibrational parameters, and a translational driver configured to provide operative translational instructions dictating translational movement;
a vibrational actuator in electrical communication with said control unit and capable of generating axial vibrations according to said operative vibrational instructions received from said vibrational driver; and
a translational motor in electrical communication with said control unit and capable of moving said insertion assembly along an insertion axis to imbed said neural implant in the target neural tissue upon receiving said operative translational instructions from said translational driver.

21. The electrode placement system of claim 20, wherein said vibrational actuator is configured to generate vibrations along said insertion axis in the range of 0.05-0.50 mm.

22. The electrode placement system of claim 20, wherein said translational motor is configured to move said insertion assembly and implant along said insertion axis at displacements in the range of about 100 μm to 20 cm and speeds in the range of 0.1 μm/s-1 m/s.

23. The electrode placement system of claim 20, further comprising a frame, wherein said vibrational actuator and said translational motor are moveably secured to said frame, and wherein said frame is one of a tabletop structure and a handheld housing.
